(19) United States
(12) Patent Application Publication
(10) Pub. No.: US 2003/0162178 A1
(43) Pub. Date: Aug. 28, 2003

(54) Variable microarray and methods of detecting one or more anlaytes in a sample

(76) Inventor: David O'Hagan, Canton, MI (US)

Correspondence Address: BRINKS HOFER GILSON & LIONE, P.O. BOX 10395, CHICAGO, IL, 60611, US

(21) Appl. No.: 10/082,789

(22) Filed: Feb. 25, 2002

(51) Int. Cl. C12Q 1/68; C12M 1/34

(52) U.S. Cl. 435/6; 435/287.2

(57) ABSTRACT

A variable microarray and methods of detecting the presence of one or more analytes in a sample are provided. In a preferred embodiment, the microarray includes a plurality of microspheres disposed on a substrate in any random order. The microspheres are individually identifiable by a color-based address. Analyte binding entities on the microspheres bind analytes if present in a test sample. An indication of binding, such as fluorescence, can be correlated with the appropriate color-based address to determine the identity of the analyte-binding entity and analyte giving rise to the indication of binding.

### FIELD OF THE INVENTION

The present invention relates to microarrays for detecting the presence of one or more anlaytes in a test sample. More particularly, the invention relates to variable position microarrays and imaging techniques using the microarrays to detect the presence of analytes in a sample.

### BACKGROUND OF THE INVENTION

Microarrays provide a powerful tool that enables the detection of one or more anlaytes in a sample. Conventional microarrays typically include a substrate with a plurality of analyte binding entities arranged on the substrate in a grid pattern. Each analyte binding entity has a unique location relative to all other such entities within the microarray. The binding of an analyte to a specific analyte binding entity, which is typically detected through chromogenic or other known indicating means, is resolved to the grid location of the analyte binding entity. The identity of the analyte can be determined by comparing the grid location with a key, such as a look-up database containing information relating to the analyte binding entity and the specific analyte to which the entity binds.

Typically, the analyte binding entity is a molecule capable of binding specifically to a particular analyte. For example, antibodies, polynucleotides, and other binding molecules are used to capture specific analytes. Accordingly, conventional microarrays comprise a plurality of such molecules deposited and secured to a substrate. Due to the relatively small size of these binding entities, many different entities can be included in any given microarray. U.S. Pat. No. 6,156,501 to McGall et al. for ARRAYS OF MODIFIED NUCLEIC ACID PROBES AND METHODS OF USE describes a representative conventional microarray. A plurality of oligonucleotide probes are secured to a substrate in a grid-like pattern, giving each particular probe a fixed and specific X,Y position on the grid. The substrate can include up to 10⁶ oligonucleotide probes at densities up to 10³ oligonucleotides per cm².

Unfortunately, conventional microarrays that have analyte binding entities directly deposited onto the substrate require intricate manufacturing processes. The molecules must be precisely positioned onto the substrate such that a suitable grid pattern is created. Also, delicate chemistries may be required to adequately bind the entities to a substrate. Indeed, many elaborate manufacturing techniques have been described. For example, U.S. Pat. No. 6,101,946 to Martinsky for a MICROARRAY PRINTING DEVICE INCLUDING PRINTING PINS WITH FLAT TIPS AND EXTERIOR CHANNEL AND METHOD OF MANUFACTURE describes methods and apparatuses for printing the analyte binding entities onto a substrate. The apparatus uses a plurality of printing pins arranged by a holder in a specific pattern with regular spacing. The tips of the pins are flat and contain a channel that holds a sample containing an analyte binding entity. During microarray manufacturing, the holder and attached plurality of pins are placed in samples to fill the channels with the binding entities. Then, the holder is moved toward the substrate, and direct contact between the pins and substrate results in the transfer of each binding entity onto the substrate. The binding entities are thereby arranged in a pattern identical to that of the pins in the holder.

As a result of these and other properties of conventional microarrays, numerous drawbacks exist in the prior art. For example, due to the complex nature of the manufacturing process, conventional microarrays capable of detecting large numbers of different analytes cannot easily be fabricated in a general research laboratory. Thus, a researcher is limited to microarrays offered from the various commercial sources, which may or may not include binding entities for the anlaytes of interest to a particular researcher. Customization, i.e., designing a microarray for a particular analyte or group of analytes, is typically expensive because it requires specialized use of the manufacturing equipment and processes.

Considering these and other defects of the prior art, there is a need for a microarray that avoids the complex manufacturing issues associated with conventional microarrays and that is easily customized for the needs of individual researchers.

### SUMMARY OF THE INVENTION

The present invention provides a microarray apparatus that is not manufactured in the conventional sense. The microarray uses microspheres arranged in a random or arbitrary pattern to avoid the need for delicate manufacturing methods. Also, the microarray can be easily customized for the needs of a given researcher.

Microarrays according to the present invention do not maintain analyte-binding entities in a fixed grid pattern on a substrate. Rather, they utilize a plurality of microspheres, each of which has a color-based address that distinguishes it from at least one other microsphere in the plurality. The microspheres are arranged in a random order on a substrate, i.e., in any order or pattern without regard to specific X,Y location on the substrate. The identity of a particular binding entity, and thus the corresponding analyte, is detected by correlating an indication of binding, such as fluorescence, with a particular color-based address. This is done by resolving the location on the substrate that corresponds to the indication of binding, and determining the color-based address of the microsphere present at that location. The color-based address, i.e., the microsphere, can be located at any point on the substrate.

Thus, microarrays according to the present invention differ significantly from those of the prior art in at least the following manner. Conventional microarrays contain analyte-binding entities assigned to a specific X,Y location prior to analysis with the microarray. In contrast, analyte binding entities in microarrays according to the present invention are not assigned to a particular X,Y location until after analysis has been initiated.

In one embodiment, the microarray of the present invention comprises a substrate and a plurality of microspheres arranged on the substrate in any random pattern. Each microsphere contains a color-based address, and an analyte binding entity on its surface. The color-based address allows for identification of a microsphere by an appropriate imaging technique. By correlating an indication of analyte binding to the binding entity on a microsphere, such as fluorescence, with a color-based address, a researcher can detect the presence of a specific analyte in a sample exposed to the microarray. Furthermore, by using a library of microspheres having different analyte specificities, with each particular analyte specificity associated with a particular color-based address, a researcher can detect the presence of multiple anlaytes in a sample. This can be done by detecting indications of binding that occur at any location(s) on the microarray and then correlating the detected indications with the color-based addresses for the microspheres present in the appropriate locations.

The use of microspheres enables a researcher to customize a microarray for detection of analytes of particular interest. For example, a researcher can attach analyte binding entities specific for a particular analyte or group of analytes to a microsphere or a library of microspheres. This avoids complex manufacturing processes because the researcher can utilize conventional microsphere binding chemistries. Thus, a researcher can easily fabricate a custom microarray by binding the analytes of interest to a library of microspheres with appropriate color-based addresses.

The present invention also provides methods of detecting the presence of one or more analytes in a test sample. In one embodiment, the method comprises exposing a library of microspheres having color-based addresses to a test sample. Each microsphere has an analyte-binding entity on its surface and a color-based address. During exposure, the analyte binding entities on the microspheres bind to their corresponding analytes, if present in the sample. Following binding, the method includes distributing the microspheres in a random or arbitrary pattern onto a substrate; detecting any indications of binding between analytes and analyte binding entities; detecting any color-based addresses of the microspheres; and correlating the indications of binding with the color-based addresses of the appropriate microspheres. The correlating of indications of binding with appropriate color-based addresses can be accomplished using location information of the indications and addresses on the microarray. The identity (ies) of analyte(s) that are bound to the microsphere(s) during the analysis can be determined by consulting a key, such as a database, and querying for the analyte, the analyte binding entity, or the color-based address. Furthermore, the identity of the bound analyte(s) can be associated with information, such as fluorescence intensity, that provides an indication of the quantity of analyte(s) present in the sample. A presence or absence, or quantifier, of an indication of binding for a particular analyte can then be obtained providing a researcher or clinician with information specific for one or more analytes.

While the invention is described in the appended claims, additional understanding of the invention can be gained from the following detailed description of the invention with reference to the appended drawings.

### BRIEF DESCRIPTION OF THE DRAWINGS

FIG. 1 is a perspective view of a variable microarray according to a preferred embodiment of the present invention.

FIGS. 2A, 2B, and 2C illustrate perspective views of various alternative embodiments of the present invention.

FIG. 3 is a schematic of a detection assay utilizing a microarray according to the present invention.

### DETAILED DESCRIPTION OF PREFERRED AND ALTERNATE EMBODIMENTS

### OF THE PRESENT INVENTION

The following description of preferred embodiments and methods provides examples of the present invention. The embodiments discussed herein are merely exemplary in nature, and are not intended to limit the scope of the invention in any manner. Rather, the description of these preferred embodiments and methods serves to enable a person of ordinary skill in the relevant art to make, use and perform the present invention.

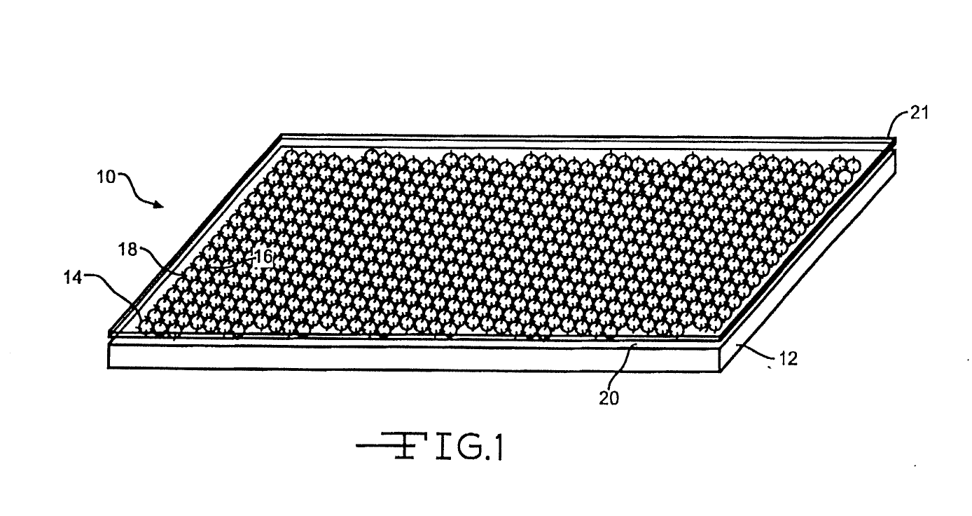

FIG. 1 illustrates a preferred embodiment of a variable microarray according to the present invention. The microarray 10 includes a substrate 12 and a plurality of microspheres 14. Individual microspheres 14 have a specific color-based address 16 that identifies a specific blend of color dye(s) associated with the microsphere 14. An analyte-binding entity 18 is present on an exterior surface of the microsphere 14, allowing the microsphere 14 to bind to an analyte of interest (not illustrated in FIG. 1).

Microspheres have previously been used in a variety of scientific applications, including chemical synthesis and separation and/or purification methods. The preparation and use of standard microspheres is known to those skilled in the art. Indeed, microspheres of various sizes and materials are readily available from several commercial sources. For example, microspheres composed of natural materials, such as agarose and cellulose, as well as synthetic materials, such as glass, polystyrene, nylon, and other polymeric materials, are readily available. Essentially any solid support that allows light to transmit through the microsphere and is able to retain the spectrally absorptive color dyes of the color-based address can be used in the present invention. Typical microspheres vary in size from approximately 0.2 μm to 200 μm in diameter.

Microspheres for use in the present invention can be prepared by using conventional microspheres known in the art. For example, conventional microspheres can be doped with a blend of absorptive color dyes to create the color-based address. The microspheres can be made by polymerizing the solid support and then doping it with the dyes of the color-based address or by any other suitable method.

Due to its ease of handling, ready availability, and ability to efficiently bind a variety of analyte-binding entities, polystyrene is a preferred material for microspheres for use in the present invention. Alternatively, of course, the microspheres can be composed from any suitable material. Also preferable, microspheres for use in microarrays according to the present invention preferably comprise substantially spherical particles that individually have a diameter of between approximately 5 μm and 200 μm. More preferably, the microspheres have a diameter of approximately 100 μm. The 100 μm size represents a balance between size and signal to noise ratio for color detection when the color-based address of the microsphere is being determined. These preferred diameters provide microspheres of a size that permits attachment of analyte-binding entities to the surface and analysis using the apparatus and methods of the present invention, as described below. It should be noted that while a spherical shape is preferred, any suitable shape can be utilized for the microspheres. Spherical shapes are preferred because they offer greater surface area than other shapes, such as disks. This allows a greater quantity of analyte-binding entities to be attached to the solid support, thereby allowing the solid support to bind a greater quantity of analyte.

As indicated above and shown in the figure, microspheres 14 in accordance with the present invention include a color-based address 16. The color-based address 16 of an individual microsphere 14 provides a unique identifier that distinguishes a particular microsphere 14 from other microspheres in a particular library.

Preferably, the color-based address 16 is a value that represents the concentration and/or optical properties of one or more color dyes doped into the substrate provided by the microsphere 14. Suitable dyes for use in the present invention include those detailed in U.S. Pat. No. 5,585,469 to Kojima et al. for DYEING AGENT HAVING AT LEAST TWO DYES FOR STAINING A BIOLOGICAL SAMPLE AND STAINING METHOD EMPLOYING THE DYEING AGENT. Particularly preferable, a blend of multiple dyes is used. The dyes used preferably have mutually independent spectral features. An ideal set of dyes for making the color-based addresses in a particular library of microspheres would cause spectral absorptance over narrow non-overlapping bands. For example, one dye might only absorb light in the spectral range between 500 nm and 550 nm, while the other dyes would operate similarly in non-overlapping spectral regions. The amount of spectral absorptance for each band, which depends on the concentration of a particular dye within the microsphere, would represent the color-based address. The dyes chosen are thus preferably optimized to ensure spectral independence of the dyes used in the color-based addresses of a particular library of microspheres for a particular microarray.

Particularly preferable, a blend of four dyes (preferably red, blue, green and yellow) is doped throughout the microsphere 14. That is, it is preferred that each microsphere 14 include a blend of dye(s) throughout its entirety. As used herein, “blend” refers to a mixture of dyes that provides a uniform distribution of a color that results from the mixture. Alternatively, any suitable number of dyes can be utilized.

Also preferable, the dyes chosen have peak transmittance, reflectance, or absorption wavelengths that, as a group, eliminate or minimize overlap of these values between individual dyes.

The color-based address 16 of an individual microsphere 14 is detected by passing broadband light, such as white light, through the microsphere 14 and measuring the transmittance, reflectance, or absorption of light, as appropriate, of specific wavelengths of light by the dye(s) doped into the microsphere 14. The wavelengths either transmitted, reflected or absorbed will depend on the presence and concentration of the dye(s) in the microsphere 14. For example, in a preferred embodiment, a higher concentration of a particular dye present in a microsphere will result in more light of the appropriate wavelength being absorbed. In this embodiment, an indication of the dye concentration(s) and/or absorption values comprise the color-based address 16.

Thus, the microspheres 14 of the present invention are distinguishable from each other based on two parameters: the presence or absence of particular dyes and the concentration of the dyes. The color-based address 16 preferably represents a value assigned to an individual microsphere 14 based on these properties. Preferably, ten different concentration levels of four different, spectrally independent dyes are used in the microspheres 14. The concentrations used will vary discretely to produce discretely measurable differences in absorptance for each dye combination. Suitable concentration increases are those that allow for accurate discrimination between concentration levels in accordance with the present invention. The use of four different dyes, each with 10 different concentrations (and thus 10 different absorptance levels) allows for the achievement of 10,000 unique color-based addresses. That is, using this arrangement, 10,000 microspheres, each having a unique identity, can be prepared. As a consequence, a single library of microspheres can be used to analyze up to 10,000 different analytes. This allows for the preparation of a customized microarray that can detect up to 10,000 different analytes.

The color-based address of a particular microsphere is determined using conventional spectrophotometric equipment and techniques. For example, using a transmission spectrophotometer, the relative absorptance of the microsphere can be determined at the appropriate wavelengths. These instruments typically provide percent absorptance values. Thus, with no dye present, 0% relative absorptance (100% relative transmittance) may be observed. The relative absorptance for that dye at the next discrete absorptance level might yield a measurement of 10% relative absorptance, and the next level 20%, and so on. Specific values of the transmittance can be set to match properties of the transmittance spectrophotometer. Furthermore, the number of discrete concentration and absorptance levels that can be discerned can be optimized based upon properties and capabilities of the transmission spectrophotometer.

It should be noted that other suitable equipment and techniques, such as absorption spectrophotometers, could be utilized in determining the color-based addresses.

Various types of analyte-binding entities 18 can be present on the surface of the microsphere 14. For example, the analyte-binding entity can comprise a single-stranded polynucleotide probe, a double-stranded polynucleotide probe, a monoclonal antibody or polyclonal sera, and a drug compound. It is important to note, however, that these illustrations are exemplary in nature and are not intended to limit the invention in any manner. Thus, it will be appreciated that the analyte-binding entity 18 can comprise any molecule or substance that can be attached to the microsphere 10 and can bind an analyte of interest in a chemically specific manner. Generally, the use of binding entities that bind analytes non-specifically is not desired.

As illustrated in FIG. 1, the substrate 12 is preferably an article having a support surface 20 onto which the microspheres 14 can be disposed. Any suitable surface can be utilized as the substrate 12. To facilitate detection of analytes using methods according to the present invention, the substrate 12 is preferably formed of a material that allows transmittance of light with minimal interface. Examples of suitable materials include glass and various plastics. Preferably, the substrate 12 comprises a conventional glass microslide known to those skilled in the art, such as conventional glass microscope slides. Preferred dimensions for the substrate 12 are 2 cm wide×7 cm long b 0.1 cm height.

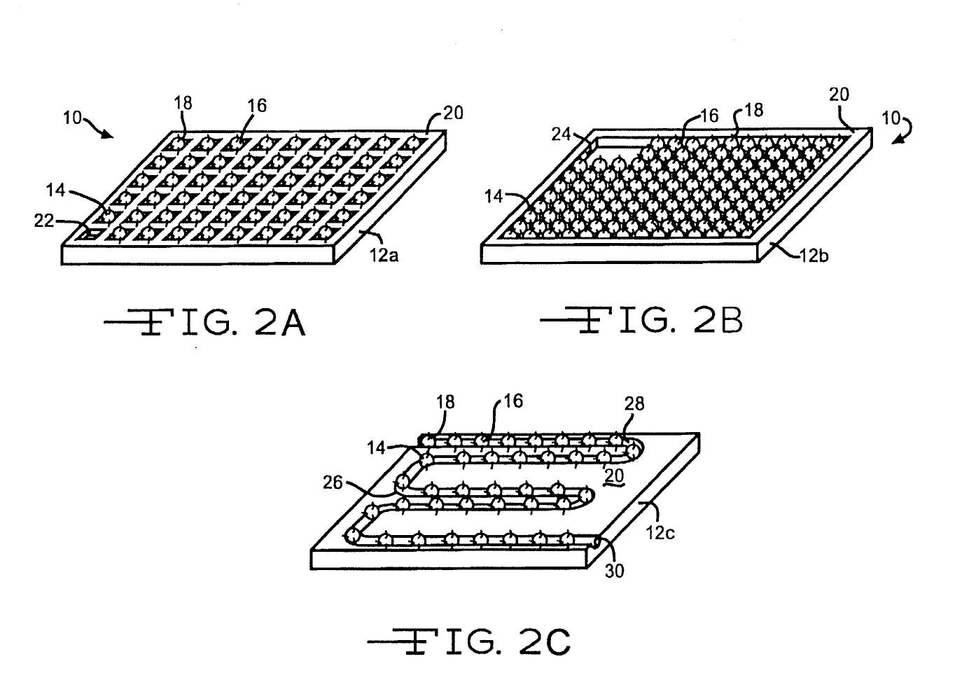

FIGS. 2A, 2B, and 2C illustrate a series of alternative forms for the substrate 12. FIG. 2A illustrates a substrate 12a that defines a series of wells 22. Each well 22 is able to receive one or more microspheres 14. Preferably, each well 22 receives a single microsphere 14. This configuration of the substrate 12a provides stability to the microarray 10 during analyte detection procedures. FIG. 2B illustrates a substrate 12b that defines a single recess 24 for receiving a plurality of microspheres 14. The recess 24 preferably mimics the configuration of the substrate 12b, thus maximizing the space available for microspheres 14. This configuration of the substrate 12b also provides stability to the microarray 10 because, during use, a sufficient number of microspheres 14 are packed into the recess 24 such that no movement occurs during analysis.

FIG. 2C illustrates a substrate 12c that defines a channel 26 or receiving a plurality of microspheres 14. The channel 26 is preferably sufficiently wide to allow movement of microspheres 14 along the channel, but also is preferably narrow enough to only allow a single microsphere 14 in any given width of the channel 26. Furthermore, channel walls 28 are preferably sufficiently high such that the distance between channel bottom 30 and support surface 20 exceeds the height of microspheres 14. This allows the use of a cover on the microarray 10 that does not mechanically interfere with the microspheres 14.

The channel 26 of the substrate 12c in FIG. 2C allows the microarray 10 to be connected to a fluidics system that can facilitate batch processing of microspheres 14. For example, the channel 2b can extend from a first edge of the substrate to a second edge. This configuration allows the microarray 10 to be attached to an apparatus outputting a stream of fluid containing a plurality of microspheres 14. The microspheres 14 can be directed through the channel 26 by applying a pressure to the stream. Once the channel 26 contains a sufficient number of microspheres, analyte detection analysis can be conducted. Preferably, the stream of microspheres remains still while analysis occurs. After the microspheres 14 in the channel 26 are analyzed, the fluidics device can inject a second plurality of microspheres into the channel 26 for analysis, thereby ejecting the first plurality of microspheres 14. This arrangement can facilitate automated processing of test samples.

To minimize any interference in the imaging procedures, as described below, the microspheres 14 are preferably packed onto the substrate 12 in a monolayer such that no unintended movement of microspheres 14 occurs. Thus, in the embodiment illustrated in FIG. 2A a single microsphere 14 is preferably placed into a single well 22. In the embodiments illustrated in FIGS. 2B and 2C, a cover 21 (illustrated in FIG. 1) can be positioned above the monolayer to retain the microspheres 14 in position. Of course, the cover 21 can be utilized in any embodiment of the invention, and can be advantageous in that it retains the microspheres 14 in position. The cover 21 can be any suitable material, so long as it does not interfere with the imaging techniques of the detection method. Preferably, the cover 21 is formed of the same material as the substrate. Particularly preferable, the cover comprises a conventional glass microscope slide coverslip known to those skilled in the art.

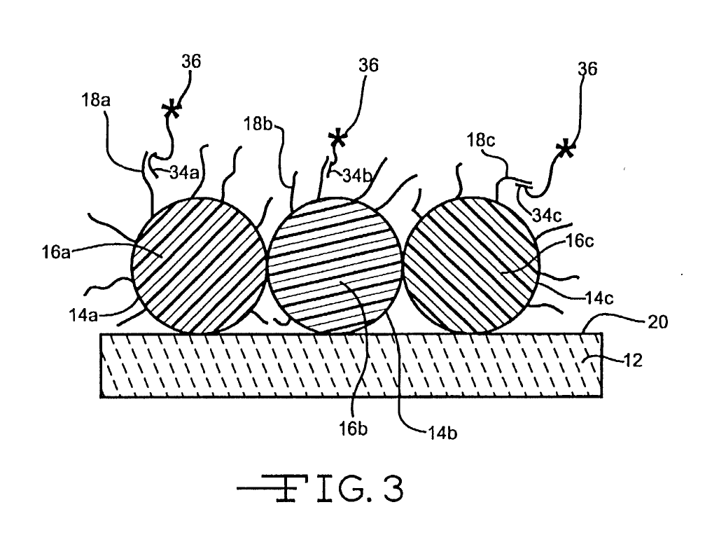

FIG. 3 illustrates a schematic of an analyte detection assay using a microarray 10 according to the present invention. In the assay, a plurality of microspheres 14 are disposed on a substrate 12 as described above. Also as indicated above, each microsphere 14 contains a color-based address 16 and an analyte binding entity 18. The analyte binding entity 18 binds to the analyte 34 which has an attached indicator 36.

As best illustrated in FIG. 3, the analyte-binding entity 18 present on the surface of the microsphere 14 binds, in a chemically specific manner, a corresponding analyte 34, if present in the sample being evaluated. Thus, the specific analyte bound will depend on the analyte-binding entity 18 present on each microsphere 14. Since a variety of analyte binding entities, such as 18a, 18b, and 18c, can be used in a single microarray 10, binding of various appropriate analytes, such as 34a, 34b, 34c, can occur in the microarray 10. For example, if the analyte-binding entity 18a is a single-stranded DNA probe, it will bind an analyte 34a comprising a single-stranded piece of DNA that contains sufficient base homology with the probe to biochemically bind the probe. Thus, the analyte is a molecule or other substance present in a sample being evaluated that is able to bind, in a chemically specific manner, to the analyte binding entity present on the surface of a microsphere. Suitable analytes include polynucleotides such as mRNA and cDNA, proteins, antigens, sugars, whole cells, chemical species, cell-bound receptors, cytokines, metabolites, drugs, and drug metabolites.

To allow detection of this binding, the analyte(s) 34 can be labeled with an indicator 36, such as a fluorescent tag. Fluorescent tags and other indicators are commonly used in the art as a tool for identifying a particular entity, and one skilled in the art will be familiar with their selection and use. A variety of fluorophores can be used as the tag. Preferred fluorescent tags include Fluorosceine Isothiocyanate (FITC), Cy3, and Cy5. Alternatively, any suitable fluorescent tag can be utilized. Fluorescent tags can be added to the analytes present in a sample according to methods known in the art.

The present invention also provides methods of detecting one or more analytes in a sample. The methods utilize variable microarrays according to the present invention. A preferred embodiment of the invention comprises exposing a library of microspheres in accordance with the present invention to a sample of interest. The exposure is preferably conducted in an environment that facilitates binding between the analyte-binding entities and the analytes. Thus, the exposing preferably occurs in a liquid environment. Temperature and other conditions can be optimized based upon the binding characteristics of the analyte-binding entities and the analytes.

The library of microspheres contains at least one microsphere that is distinguishable from at least a second microsphere based upon its color-based address. Particularly preferable, the library contains a plurality of microspheres, each of which is distinguishable from all other microspheres in the library based upon its color-based address. Furthermore, each microsphere preferably has a unique analyte-binding entity on its surface (best illustrated as analyte binding entities 18a, 18b, and 18c in FIGS. 2A, 2B, and 2C, respectively).

During exposure of the microspheres to the sample, the analyte binding entities on the microspheres bind to the appropriate, i.e., chemically specific, analytes if they are present in the sample. Next, the microspheres are distributed across a substrate to form a variable microarray. The microspheres are distributed randomly or arbitrarily across the substrate, i.e., without regard to the location of individual microspheres on the substrate. The distributing can be accomplished using standard laboratory liquid-handling techniques, such as dispensing liquid with a pipette type dispenser.

Next, indications of binding between analytes and analyte-binding entities are detected. This step depends on the type of indicator used. For example, if the analytes are labeled with fluorescent tags, the detection comprises detecting fluorescence associated with the microspheres due to analyte binding. Fluorescence detection techniques are known to those skilled in the art, and any suitable technique and detection apparatus can be used. Data, such as fluorescence intensity, relating to the indication of binding is then associated with the location on the microarray at which the indication of binding resides. The location can comprise any suitable location identifier, such as an X,Y coordinate, well identifier, or the like.

The color-addresses of the microspheres are also determined. As discussed above, this is typically accomplished by using conventional spectrophotometric equipment and techniques to measure the absorptance or transmittance of a microsphere at appropriate wavelengths. Typically, a broadband light source is directed through the microsphere and a reading of relative spectral absorptance is taken. In this step, many optical configurations can be utilized. For example, one suitable configuration involves flood illuminating the substrate of the microarray with a broadband light source and then measuring the relative spectral transmittance using a detector array located in the image plane of an imaging system. Such measurements are often accomplished by placing specific known transmission filters in the optical train between the microspheres and the imaging detector. Digital output from the detector array then yields measurements of the relative spectral transmittance which, in turn, indicates the color-based address for a particular microsphere(s).

The determination of color-based address(es) can be performed for all microspheres in the microarray, or only for those microspheres positioned on the substrate at the location(s) that corresponds to an indication of binding (e.g., fluorescence). In one embodiment, the color-based address(es) at the appropriate location(s) are determined. However, it may be desirable to determine all color-based addresses in the microarray regardless of the presence of an indication of binding. In this embodiment of the method, the indications of binding, if any, are detected and their location on the microarray are recorded. Color-based addresses are then determined for all microspheres in the microarray, and the location of each address is recorded. Finally, correlation of these data is performed as indicated below.

Once indications of binding and color-based addresses are determined, these data are correlated based upon location on the microarray. That is, a particular indication of binding is correlated with the color-based address that was determined at the same location on the microarray. For example, if at a particular X,Y position or well, fluorescence is detected at Level A, and color-based address 4617 is determined to be at that same location, these data are combined, based on their being associated with the same location on the microarray, to indicate that Level A of fluorescence was detected for color-address 4617. The process can then include determining the identity of the analyte-binding entity (or analyte) that is associated with the particular color-based address. This can be accomplished by consulting a look-up table, such as a database, querying the table by color-based address, and obtaining the desired identity.

For example, a look-up table may indicate that the microsphere having color-based address 4617 has the insulin receptor as the analyte-binding entity on its surface. By querying the look-up table by the address, a researcher can associate the level of fluorescence, Level A, with the particular analyte-binding entity, insulin receptor, or analyte, insulin. Furthermore, by comparing the indicator, such as a level of fluorescence, to a standard, the researcher can determine the quantity of analyte present in the sample. This process can be conducted for each unique color-based address in the library of microspheres used in the microarray.

It should be noted that in the methods of detecting one or more analytes in a sample according to the present invention, the exposing the microspheres to the sample can occur either before, during or after distributing the microspheres onto the substrate.

The present invention also comprises kits of materials that allow a researcher to fabricate variable microarrays customized to particular analytes. In a preferred embodiment, the kits include a substrate as discussed above and a library of microspheres. Each microsphere is preferably distinguishable from at least one other microsphere in the library based upon a color-based address as discussed above. Particularly preferable, each microsphere is distinguishable from all other microspheres in the library based upon the color-based address.

A researcher can fabricate a custom microarray according to the present invention by attaching analyte-binding entities of interest to the microspheres, and recording a correlation between the color-based address(es) and the appropriate analytes in a look-up table. The microspheres are then randomly distributed onto a substrate, either before, during or after exposing the microspheres to a sample of interest. The custom microarray can then be used in the methods of the present invention.

### EXAMPLE 1

A variable microarray for detecting known cancer specific proteins can be fabricated as follows. A library of monoclonal antibodies, each of which binds a particular cancer-specific protein, can be attached to a library of microspheres. The antibodies are bound in a manner such that each antibody is bound to one more microspheres having a unique color-based address. The library of microspheres can then be exposed to a fluorescent protein sample prepared from a sample, such as a protein component of a patient biopsy. During exposure, the antibodies are allowed to bind to their antigens, i.e., cancer-specific proteins, if the antigens are present in the sample. Next, the microspheres are randomly or arbitrarily applied to a substrate as described above. Fluorescence signals, or lack thereof, are detected as appropriate. The detected fluorescence, which indicates binding between the analyte-binding entity and the analyte (antibody and antigen) is associated with the appropriate microarray location indicators, such as X,Y coordinate(s) or well identifiers. The color-based addresses at these locations are determined. The fluorescence data and color-based addresses are correlated by combining the information based upon being associated with the same location in the microarray. Then, the identity of the cancer-specific protein(s) present in the sample is resolved by consulting an appropriate database. This allows a researcher or clinician to identify the cancer-specific proteins present in the sample and proceed accordingly.

### EXAMPLE 2

This example is identical to Example 1, except during analysis, color-based addresses of all microspheres in the microarray are determined, regardless of whether an indication of binding was detected at the location of the microsphere. Correlation of indications of binding and color-based addresses is still conducted based upon location on the microarray. For locations at which no indication of binding was detected, a null, zero, or background entry is assigned to the color-based address. By determining all color-based addresses, regardless of indications of binding, a more thorough evaluation of the sample can be conducted.

All references cited in this disclosure are hereby incorporated into this disclosure in their entirety, except to any extent to which they contradict any statement or definition made herein.

The foregoing disclosure includes the best mode devised by the inventor for practicing the invention. It is apparent, however, that several variations in the apparatuses and methods of the present invention may be conceivable by one skilled in the art. In as much as the foregoing disclosure is intended to enable one skilled in the pertinent art to practice the instant invention, it should not be construed to be limited thereby, but should be construed to include such aforementioned variations.

1. A microarray for detecting the presence of one or more analytes in a sample, the microarray comprising:
a substrate defining a support surface;
a plurality of microspheres randomly distributed on the support surface, each microsphere comprising:
a solid support having an exterior surface;
a color-based address comprising one or more dyes contained within the solid support and adapted to identify an individual microsphere; and,
an analyte-binding entity attached to the exterior surface which binds one of said analytes.

2. A microarray in accordance with claim 1, wherein the solid support comprises a member selected from the group consisting of polystyrene, nylon, and glass.

3. A microarray in accordance with claim 1, wherein the one or more dyes comprises a plurality of dyes, each dye of the plurality of dyes capable of absorbing light at a wavelength distinct from wavelengths at which all other dyes in the plurality of dyes are capable of absorbing light.

4. A microarray in accordance with claim 3, wherein the plurality of dyes comprises four dyes.

5. A microarray in accordance with claim 1, further comprising a cover disposed on the substrate above the plurality of microspheres.

6. A microarray in accordance with claim 1, wherein the plurality of microspheres includes a first microsphere distinguishable from at least a second microsphere based upon the color-based address.

7. A microarray in accordance with claim 1, wherein the plurality of microspheres includes a first microsphere distinguishable from all other microspheres in the plurality of microspheres based upon the color-based address.

8. A microarray in accordance with claim 1, wherein the support surface defines a plurality of wells.

9. A microarray in accordance with claim 8, wherein each well in the plurality of wells is adapted to receive only a single microsphere from the plurality of microspheres.

10. A microarray in accordance with claim 1, wherein the support surface defines a recess adapted to receive at least two microspheres of the plurality of microspheres.

11. A microarray in accordance with claim 10, wherein the recess is adapted to receive all microspheres in the plurality of microspheres.

12. A microarray in accordance with claim 1, wherein the support surface defines a channel.

13. A microarray in accordance with claim 12, wherein the substrate has first and second edges and wherein the channel extends from the first edge to the second edge.

14. A microarray in accordance with claim 13, wherein the channel extends from the first edge to the second edge along a non-linear path.

15. A microarray in accordance with claim 12, wherein the channel has a width sufficient to accommodate only a single microsphere form the plurality of microspheres.

16. A microarray in accordance with claim 12, wherein the channel includes channel walls, and wherein the channel walls have a height that is greater than the height of the microspheres.

17. A microarray in accordance with claim 16, wherein the plurality of microspheres is disposed in the channel; and further comprising a cover disposed on the substrate above the plurality of microspheres.

18. A microarray for detecting the presence of one or more analytes in a sample, the microarray comprising:
a plurality of microspheres randomly distributed on a support surface;
wherein each microsphere of the plurality of microspheres includes an analyte binding entity which binds one of said analytes and a color-based address.

19. A microarray in accordance with claim 18, wherein the color-based address comprises a plurality of dyes, each dye of the plurality of dyes capable of absorbing light at a wavelength distinct from wavelengths at which all other dyes in the plurality of dyes are capable of absorbing light.

20. A method of detecting one or more analytes in a sample, comprising:
providing a plurality of microspheres, each microsphere comprising a solid support having an exterior surface, a color-based address, and an analyte-binding entity attached to the exterior surface which binds one of said analytes;
exposing the plurality of microspheres to said sample;
randomly distributing the plurality of microspheres onto a substrate;
detecting indications of binding between said analytes and the analyte-binding entities on the plurality of microspheres;
associating each indication of binding with the location on the substrate of the analyte-binding entity to which said analyte has bound;
determining the color-based address of the microsphere at the location of each indication of binding; and
correlating each indication of binding with the color-based address based upon the location.

21. A method of fabricating a microarray for detecting the presence of one or more analytes present in a sample, the method comprising:
providing a plurality of microspheres, each microsphere comprising a solid support having an exterior surface and a color-based address;
attaching an analyte-binding entity capable of binding one of said analytes to the exterior surface of at least one of the microspheres in the plurality of microspheres; and
randomly distributing the plurality of microspheres onto a substrate.
